(19) United States
(12) Patent Application Publication
(10) Pub. No.: US 2015/0335261 A1
(43) Pub. Date: Nov. 26, 2015

(54) EXPANDABLE CAGE SURROUNDING A MAGNETIC RESONANCE DEVICE AND METHODS THEREOF

(71) Applicant: ASPECT IMAGING LTD., Shoham (IL)

(72) Inventor: Uri RAPOPORT, Moshav Ben Shemen (IL)

(21) Appl. No.: 14/709,512

(22) Filed: May 12, 2015

Related U.S. Application Data
(60) Provisional application No. 62/001,105, filed on May 21, 2014.
(60) Provisional application No. 62/006,919, filed on Jun. 3, 2014.

(51) Int. Cl. A61B 5/055; A61B 5/00; G01R 33/3815; G01R 33/30; G01R 33/383

(52) U.S. Cl. CPC A61B 5/055; G01R 33/307; G01R 33/383; G01R 33/3815; A61B 5/4255

(57) ABSTRACT

Devices and methods for providing an adjustable open-bore magnetic resonance device (MRD) that enable personalized accommodation for users of different sizes. The MRD includes a first main magnetic source and a second main magnetic source in a face-to-face orientation. The magnetic sources are separated by a distance that is adjustable by way of an endoskeletal height adjuster. In some embodiments, a superconducting electromagnet coiled around an oval defines the magnetic volume of interest. In such embodiments, the perimeter of the oval includes a plurality of trapezoid elements housing the coil of the electromagnet, and the trapezoid elements can be moved outwards and inwards relative to the oval's center in the plane of the oval.

### FIELD OF THE INVENTION

The present invention generally relates to a magnetic resonance imaging device (MRD) and more specifically the present invention discloses an expandable cage of an MRD for comprising objects varying in size therein its volume of interest (VOI).

### BACKGROUND OF THE INVENTION

Electromagnetism-based instruments for measuring properties of matter or identifying its composition are well known. Magnetic resonance spectroscopy is one of the principal measuring techniques used to obtain physical, chemical and structural information about a molecule. Magnetic resonance imaging (MRI) devices greatly vary in the size of their volume of interest (VOI), and the size is directly correlated with the device's cost and weight. Currently, MRI devices range from a ˜700 kg device designed to contain a mouse and up to several ton devices designed to accommodate humans. As a result, MRI devices designed for humans are costly and require specialized conditions for storage and use.

One of the many uses of MRI devices is determination of the progress of healing in patients suffering from Inflammatory Bowel Disease (IBD). IBD refers to a group of chronic or recurring inflammation-related diseases of the colon and small intestine, such as Crohn's disease or ulcerative colitis. The prevalence of IBD is ˜400 per 100,000 persons in Europe and North America (according to http://www.macc.org.mt/home/statistics.html, which is incorporated herein as a reference), occurring more in people of Caucasian and Ashkenazi Jewish origin than in other racial and ethnic subgroups Annual expenses of IBD disease medication are estimated to be on the order of 67B$; however, these medications are only aimed at relieving the symptoms of the disease and, currently, there is no cure for the disease.

Finding a cure for IBD disease is extremely challenging as there does not appear to be any animal model for this disease. An indication of a successful treatment can be provided only with the use of MRI which enables the imaging of the patient's gut, thereby enabling following the progress of mucosal healing. However, due to the high costs and specialized requirements for human MRI devices available today, the number of patients with access to MRI imaging is rather limited.

An MRI device with a reduced size and/or costs could be achieved by adjusting the device according to the patient's size. More specifically, an expandable VOI could provide more flexibility in the design of MRI devices.

U.S. Pat. No. 7,215,231 presents an MRI device where the upper pole is movable with respect to the upper pole support located in the ferromagnetic frame. To facilitate the movement of the upper pole, shafts are provided having a first end coupled to the upper pole and a second end coupled to a motor supported by the ferromagnetic frame. This movement results in increasing the size of the VOI between opposite poles of a ferromagnetic frame facilitating access to the patient by medical personnel during a medical procedure.

However, due to the external expansion mechanism this presented MRI device cannot address the problem of device size and weight, as the outer cage is designed to fit the widest modification.

An expandable cage comprising self-fastening cage walls with an inherent expansion mechanism will provide flexibility in the size of the desired VOI and will result in a device which is both more compact and more convenient to store, fulfilling a long-felt need for a lower-cost, storage-friendly MRI device.

### SUMMARY OF THE INVENTION

It is thus one object of the invention to provide an open-bore magnetic resonance device (MRD) having length, width and height (X, Z and Y axis, respectively), comprising a first main magnetic source and a second main magnetic source arranged in a face-to-face orientation along its Y axis, defining in-between a volume of interest (VOI); at least two top extension plates extending from the first main magnetic source and at least two bottom extension plates extending from the second main magnetic source; these top and bottom extension plates are arranged in a face-to-face orientation along the MRD's Y axis; and at least two outer shell walls extending perpendicular to the extension plates, which are configured by means of size, shape and material to confine the VOI; wherein the outer shell walls comprise a height adjuster for adjusting the distance between the first main magnetic source and the second main magnetic source; the height adjuster is provided with protruding elements for interlocking with corresponding bores in the extension plates; and in addition, the height adjusters define at least one narrow configuration (YN) and at least one wide configuration (YW), such that the VOI is reversibly expandable and a personalized accommodation of a user according to his dimensions is enabled.

It is also in the scope of the present invention to provide the MRD as defined above and providing the same with at least two pole-pieces, arranged in a face-to-face orientation; wherein the first main magnetic source and the second main magnetic source are permanent magnets located on the pole-pieces, arranged in a face-to-face orientation such that a static magnetic field is generated within the VOI.

It is also in the scope of the present invention to provide the MRD as defined above and providing the same with at least six side-magnets, at least a portion of the side-magnets being superconductors or ferromagnets, and these side-magnets extend from the first and second main magnetic sources defined above and are arranged in equal groups being in a face-to-face orientation in a magnetic connection with the above mentioned extension plates, such that the overall strength of the magnetic field provided in the cage of the MRD is increased.

It is also in the scope of the present invention to provide the MRD as defined above wherein the side-magnets as defined above and the extension plates are configured as a self-fastening cage.

It is another object of the present invention to provide a method for obtaining an expandable cage of an MRD comprising steps of providing an open-bore magnetic resonance device (MRD) with length, width and height (X, Z and Y axis, respectively), the open-bore magnetic resonance device comprising: (i) a first main magnetic source and a second magnetic source arranged in a face-to-face orientation along the Y axis, defining in-between a volume of interest (VOI) and (ii) at least two outer shell walls for confining the VOI; wherein, by means of a height adjuster comprised in the outer shell walls, reversibly adjusting the distance between the first main magnetic source and the second main magnetic source, thereby enabling a personalized accommodation of a user according to his dimensions.

It is also in the scope of the present invention to provide the MRD as defined above additionally comprising a step of providing a height adjuster with protruding elements for interlocking with corresponding bores in the outer shell walls as defined above.

It is also in the scope of the present invention to provide the MRD as defined above and providing the same with a step of configuring the MRD as a self-fastening caged MRD, the outer shell walls defined above comprising an outside shell.

It is also in the scope of the present invention to provide the MRD as defined above additionally comprising a step of superimposing at least three flexi-jointed walls, thereby providing a homogenous, stable and uniform magnetic field therein.

It is another object of the present invention to provide an expandable open-bore magnetic resonance device (MRD) comprising at least one non-permanent superconducting electromagnet coiled around the perimeter of an oval defining a magnetic volume of interest (VOI), wherein the oval's perimeter comprises a plurality of trapezoid elements housing the coil of the electromagnet, and further wherein the trapezoid elements are provided with a dislocating mechanism for shifting the trapezoid elements outwards and inwards relative to the oval's center and substantially in the oval's plane, thus defining at least one narrow configuration (ON) and at least one wide configuration (OW), thereby reversibly expanding the VOI and enabling a personalized accommodation of a user according to his dimensions.

It is also an object of the present invention to provide a method for obtaining an expandable cage of an MRD comprising steps of providing an open-bore MRD comprising at least one non-permanent superconducting electromagnet, and defining a magnetic volume of interest (VOI) by coiling the electromagnet around the perimeter of an oval, wherein the above mentioned method further comprises steps of providing the oval's perimeter with a plurality of trapezoid elements housing the electromagnet coil, and further providing the trapezoid elements with a dislocating mechanism for shifting the trapezoid elements outwards and inwards relative to the oval's center and substantially in the oval's plane, thus defining at least one narrow configuration (ON) and at least one wide configuration (OW), thereby reversibly expanding the VOI and enabling a personalized accommodation of a user according to his dimensions.

It is another object of the present invention to provide a method for scoring mucosal healing parameters in an IBD patient, comprising steps of providing an open-bore magnetic resonance device (MRD), providing at least one main magnetic source defining a volume of interest (VOI) and providing the same with adjusting means for reversibly expanding the VOI, thereby enabling a personalized accommodation of the IBD patient's abdominal region according to the patient's abdominal dimensions.

Another important object of this invention is to provide a new standard of care for an IBD patient's treatment surveillance, based on the method mentioned above.

### BRIEF DESCRIPTION OF THE FIGURES

In order to better understand the invention and its implementation in practice, a plurality of embodiments will now be described, by way of non-limiting example only, with reference to the accompanying drawings, wherein

FIGS. 1 and 2 schematically illustrate partial sectional perspective side views, not to scale, of a substantially parallelepiped adjustable MRI;

FIGS. 3-5B schematically illustrate side views, not to scale, of a substantially parallelepiped adjustable MRI;

FIG. 6A-B schematically illustrates a side view, not to scale, of a use of a substantially parallelepiped adjustable MRI;

FIG. 7 schematically illustrates, not to scale, an expandable open-bore magnetic resonance device comprising a superconducting electromagnet coiled around an oval; and

FIG. 8A-B schematically illustrates, not to scale, a use of an expandable open-bore magnetic resonance device comprising a superconducting electromagnet coiled around an oval.

### DETAILED DESCRIPTION OF THE PREFERRED EMBODIMENTS

The following description is provided, alongside all chapters of the present invention, so as to enable any person skilled in the art to make use of said invention and sets forth the best modes contemplated by the inventor of carrying out this invention. Various modifications, however, will remain apparent to those skilled in the art, since the generic principles of the present invention have been defined specifically to provide an open-bore magnetic resonance device (MRD) having length, width and height (X, Z and Y axis, respectively), comprising: a first main magnetic source and a second main magnetic source arranged in a face-to-face orientation along the Y axis, defining in-between a volume of interest (VOI); at least two top extension plates extending from the first main magnetic source and at least two bottom extension plates extending from the second main magnetic source; the top and bottom extension plates arranged in a face-to-face orientation along the Y axis; and at least two outer shell walls extending perpendicular to the extension plates for confining the VOI; wherein the outer shell walls comprise a height adjuster for adjusting the distance between the first main magnetic source and the second main magnetic source; the height adjuster being provided with protruding elements for interlocking with corresponding bores in the extension plates; with the height adjusters defining at least one narrow configuration (YN) and at least one wide configuration (YW), thereby enabling reversible expansion of the VOI and enabling a personalized accommodation of a user according to the user's dimensions. Most surprisingly, the disclosed MRD is reversibly expandable by endoskeletal means, as opposed to the prior art, which cited cumbersome and unwieldy exoskeletal mechanisms only.

The term ‘magnetic resonance device’ (MRD) refers hereinafter to any Magnetic Resonance Imaging (MRI) device, any Nuclear Magnetic Resonance (NMR) spectroscope, any Nuclear Quadrupole Resonance (NQR) device and any combination thereof

The term ‘volume of interest’ (VOI) refers hereinafter to a region of uniform magnetic field of the MRD. Typically, an object to be imaged is placed within the VOI.

The term ‘height adjusters’ refers hereinafter to protruding elements located inherently in the MRD cage's outer shell walls, surrounding the VOI. The height adjusters are provided with means for adjusting their protrusion length, thereby reversibly expanding the VOI.

The term ‘expandable’ refers hereinafter to an endoskeleton expanding mechanism of the height adjusters of an MRD (100) or the expanding mechanism of the dislocating mechanism of the trapezoid elements of an MRD (200). The height adjusters are configured to expand or contract along the Y axis of the MRD (100) while the trapezoid elements are configured to move outward or inward relative to the center of the oval and parallel to the plane of the oval of the MRD (200), providing an expandable VOI in each case.

The term ‘dislocating mechanism’ refers hereinafter to the mechanism or mechanisms which move the trapezoid elements of an MRD and enable the change of size and shape of the oval.

The term ‘extension-plates’ refers hereinafter to plates connecting the main magnet's cage with the shell walls, extending in parallel to the main magnets and perpendicular to the cage's outer shell walls. The extension plates are provided with bores for interlocking with the height adjusters' protruding elements.

The term ‘self-fastening’ refers hereinafter to a strong magnetic connection between the side-magnets and the cage walls. The magnets' edges are attracted to each other such that a closed form is provided. The cage, magnetically attracted to the side-magnets, supports itself without need for another connection.

The term ‘pole-piece’ refers hereinafter to an element of high permeability material used to shape the uniformity of the magnetic flux from a permanent magnet.

The term ‘side-magnets’ refers hereinafter to permanent magnets arranged around the sides of the pole-pieces.

The term ‘trapezoid elements’ broadly refers hereinafter to any element having a polygonal cross-section and more specifically trapezoid shaped segments surrounding the perimeter of an oval in an open bore MRD where the non-permanent superconducting electromagnet of the MRD which defines the magnetic volume of interest is coiled around the perimeter of the oval. The trapezoid elements are arranged in an alternate head-to-toe fashion.

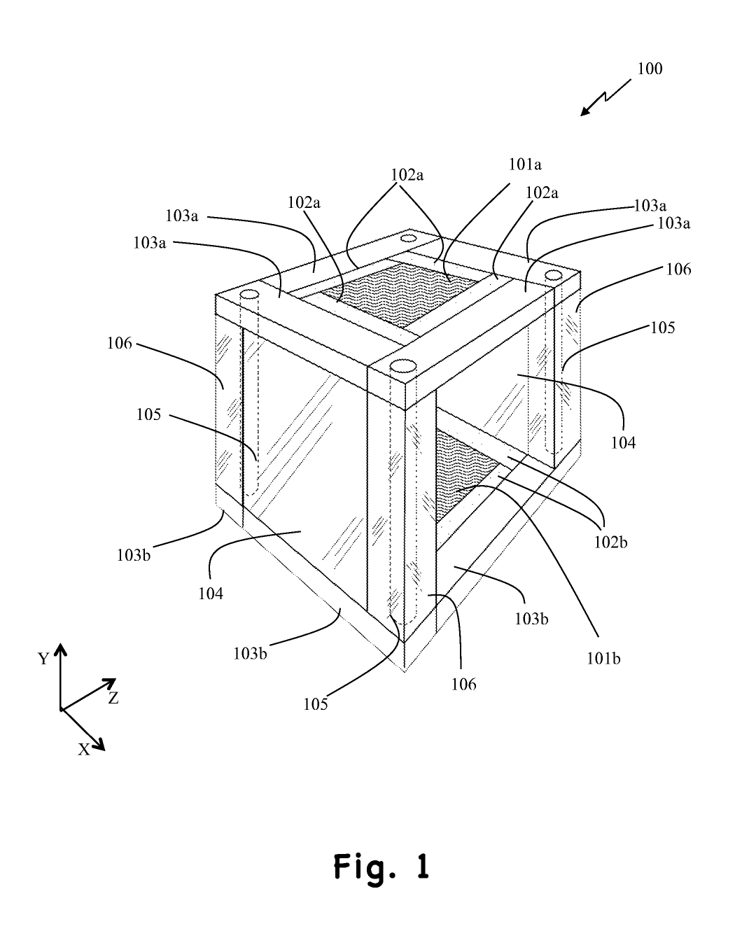

Reference is now made to FIG. 1 illustrating an open-bore magnetic resonance device (MRD) having length, width and height (X, Z and Y axis, respectively), comprising a first main magnetic source and a second main magnetic source arranged in a face-to-face orientation along its Y axis, defining in-between a volume of interest (VOI). The MRD also comprises at least two top extension plates extending from the first main magnetic source and at least two bottom extension plates extending from the second main magnetic source. These top and bottom extension plates are arranged in a face-to-face orientation along the MRD's Y axis. In addition, there are at least two outer shell walls extending perpendicular to the extension plates for confining the VOI; with the outer shell walls comprising a height adjuster for adjusting the distance between the first main magnetic source and the second main magnetic source. The height adjuster is provided with protruding elements for interlocking with corresponding bores in the extension plates and, in addition, the height adjuster defines at least one narrow configuration (YN) and at least one wide configuration (YW), thereby enabling reversible expansion of the VOI and enabling a personalized accommodation of a user according to his dimensions.

More specifically, FIG. 1 schematically presents a partial sectional perspective side view, not to scale, with respect to an axial plane of the CLOSED configuration of the 3D MRD (100), illustrating a preferred embodiment of the present invention, wherein the device has a substantially parallelepiped shape, with a first top main magnet (101a) and a second bottom main magnet (101b) arranged in a face-to-face orientation along the Y axis, defining in-between a volume of interest (VOI). Each main magnet is surrounded by four side-magnets (102a and 102b), and is further surrounded by at least two extension-plates (103a and 103b), at least two cage walls (104) comprising bores (106) which comprise at least two height adjusters (105) (shown is a configuration of four such height adjusters) such as, in a non-limiting example, expandable shafts. In FIG. 1, the height adjusters are presented in a closed configuration and are arranged in equal groups interlocking with corresponding bores located in the extension plates (103a and 103b).

Moreover, FIG. 1 enables one skilled in the art to make use of a method for obtaining an expandable cage of an MRD (100) comprising: providing an open-bore magnetic resonance device (MRD, 100) with length, width and height (X, Z and Y axis, respectively), providing a first main magnetic source (101a) and a second magnetic source (101b) arranged in a face-to-face orientation along said Y axis, defining in-between a volume of interest (VOI) with a static magnetic field and providing the same with at least two outer shell walls (104) for confining said VOI; comprising bores (106) which comprise two or more height adjusters (105), wherein by means of the two or more height adjusters (105), adjusting the distance between the first main magnetic source (101a) and said second main magnetic source (101b).

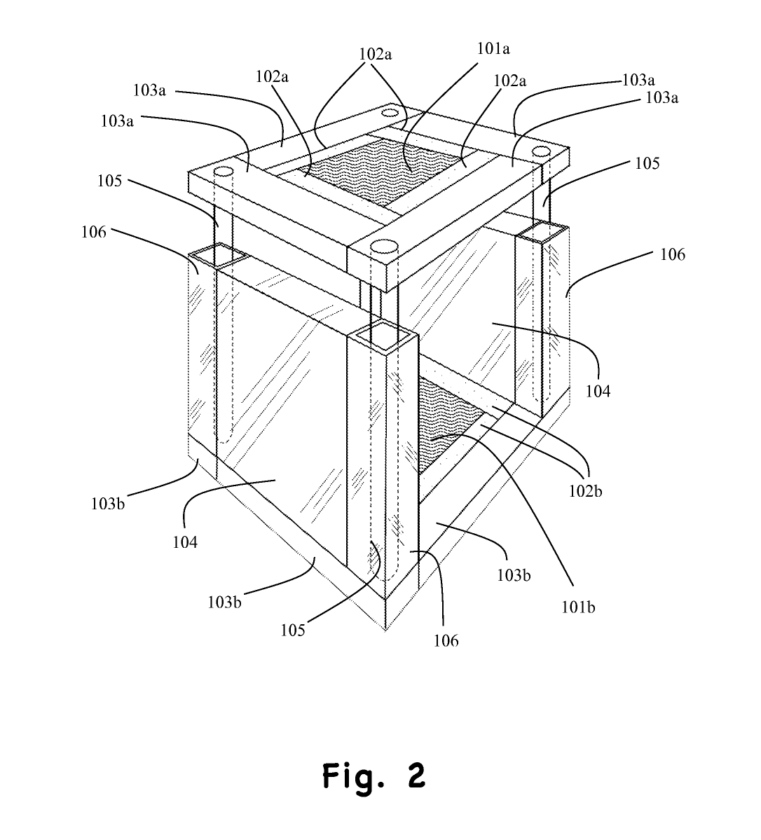

Reference is now made to FIG. 2 schematically presenting a partial sectional perspective side view, not to scale, with respect to an axial plane of the OPEN configuration of the 3D MRD (100) as presented in a CLOSED configuration in FIG. 1. FIG. 2 illustrates a preferred embodiment of the present invention, wherein the device has a substantially parallelepiped shape, with a first top main magnet (101a) and a second bottom main magnet (101b) arranged in a face-to-face orientation along the Y axis, defining in-between a volume of interest (VOI). Each main magnet is surrounded by four side-magnets (102a and 102b), and is further surrounded by at least two extension-plates (103a and 103b), and at least two cage walls (104) comprising bores (106) which comprise at least two height adjusters (105) (shown is a configuration of four such height adjusters) such as, in a non-limiting example, expandable shafts. In FIG. 2, the height adjusters are presented in an open configuration and are arranged in equal groups interlocking with corresponding bores located in the extension plates (103a and 103b).

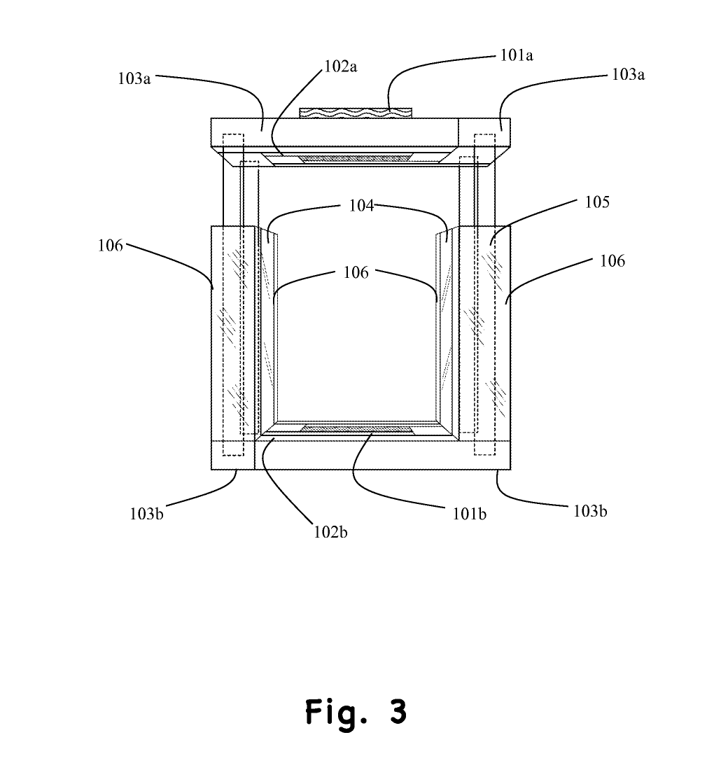

Reference is now made to FIG. 3 schematically presenting a side view, not to scale, of the OPEN configuration of the 3D MRD (100) wherein the device has a substantially parallelepiped shape, with a first top main magnet (101a) and a second bottom main magnet (101b) arranged in a face-to-face orientation along the Y axis, defining in-between a volume of interest (VOI), Each main magnet is surrounded by four side-magnets (102a and 102b), and is further surrounded by at least two extension-plates (103a and 103b), and at least two cage walls (104) comprising bores (106) which comprise at least two height adjusters (105) (shown is a configuration of four such height adjusters) such as, in a non-limiting example, expandable shafts. In FIG. 3, the height adjusters are presented in an open configuration and are arranged in equal groups interlocking with corresponding bores located in the extension plates (103a and 103b).

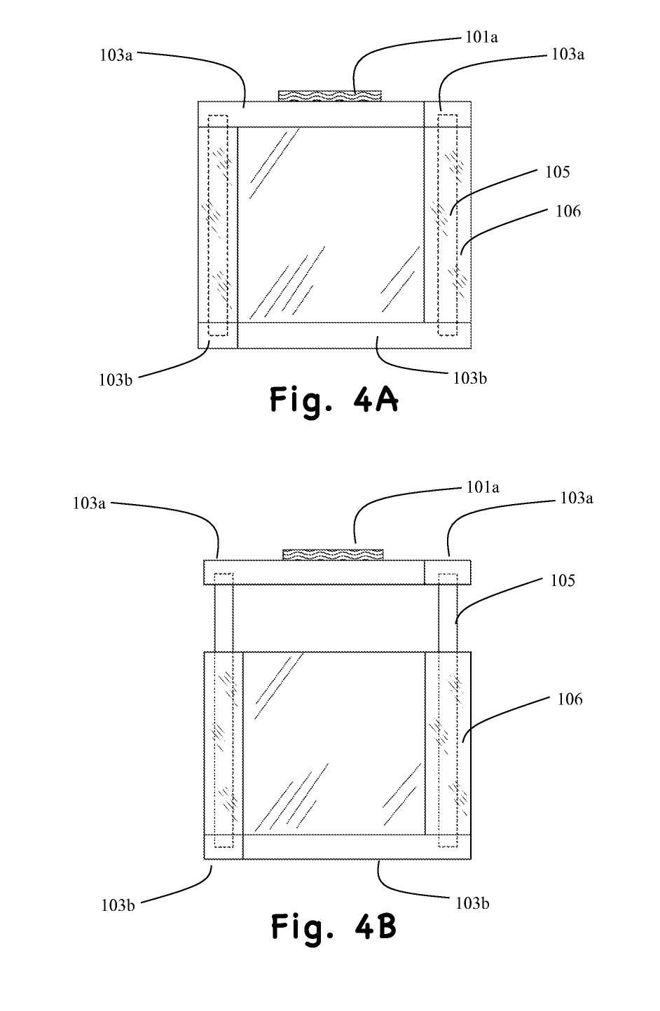

Reference is now made to FIG. 4A schematically presenting a side view, not to scale, of the CLOSED configuration of the 3D MRD (100) wherein the device has a substantially parallelepiped shape, with a first top main magnet (101a) and a second bottom main magnet (not shown) arranged in a face-to-face orientation along the Y axis, defining in-between a volume of interest (VOI), each main magnet is surrounded by at least two extension-plates (103a and 103b), and at least two cage walls (104) comprising bores (106) which comprise at least two height adjusters (105) (shown is a configuration with four such height adjusters; only two are shown). The height adjusters can be, in a non-limiting example, expandable shafts or interlocking male and female protrusions. The height adjusters are arranged in equal groups interlocking with corresponding bores located in the extension plates (103a and 103b). In FIG. 4A, the height adjusters are presented in a closed configuration.

Reference is now made to FIG. 4B schematically presenting a side view, not to scale, of the OPEN configuration of the 3D MRD (100) wherein the device has a substantially parallelepiped shape, with a first top main magnet (101a) and a second bottom main magnet (not shown) arranged in a face-to-face orientation along the Y axis, defining in-between a volume of interest (VOI). Each main magnet is surrounded by at least two extension-plates (103a and 103b), and at least two cage walls (104) comprising bores (106) which comprise at least two height adjusters (105) (shown is a configuration with four such height adjusters; only two are shown). The height adjusters can be, in a non-limiting example, expandable shafts or interlocking male and female protrusions. The height adjusters are arranged in equal groups interlocking with corresponding bores located in the extension plates (103a and 103b). In FIG. 4B, the height adjusters are presented in an open configuration.

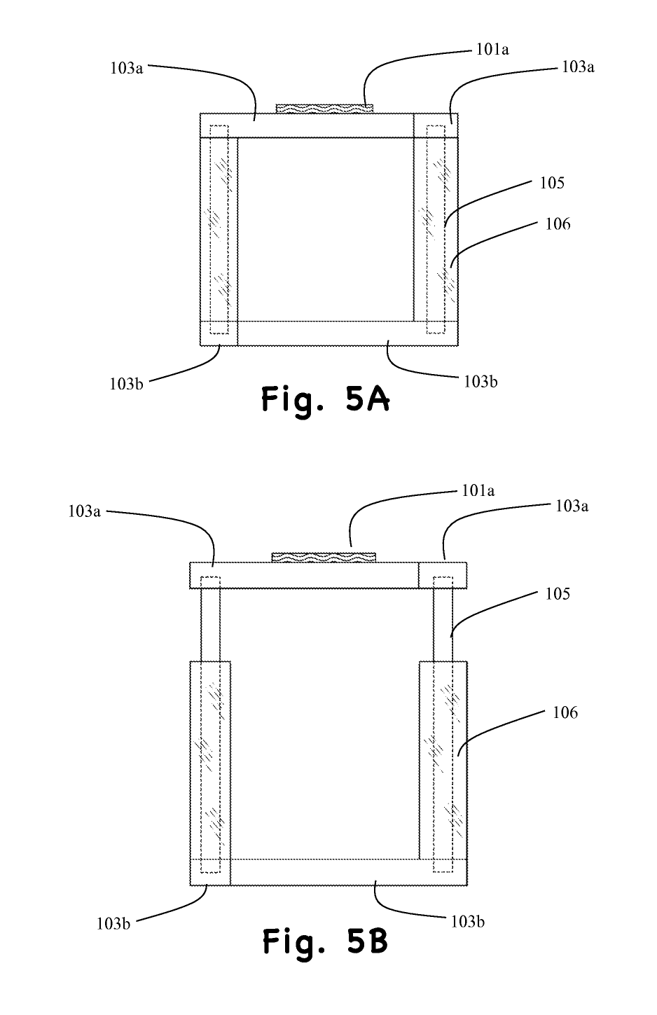

Reference is now made to FIG. 5A schematically presenting another side view, not to scale, of the CLOSED configuration of the 3D MRD (100), rotated 90 degrees in the XZ plane with respect to FIGS. 4A and 4B, wherein the device has a substantially parallelepiped shape, with a first top main magnet (101a) and a second bottom main magnet (not shown) arranged in a face-to-face orientation along the Y axis, defining in-between a volume of interest (VOI). Each main magnet is surrounded by at least two extension-plates (103a and 103b), and at least two cage walls (not shown) confining the VOI and comprising bores (106) which comprise at least two height adjusters (105) (shown is a configuration with four such height adjusters; only two are shown). The height adjusters can be, in a non-limiting example, expandable shafts, or interlocking male and female protrusions. The height adjusters are arranged in equal groups interlocking with corresponding bores located in the extension plates (103a and 103b). In FIG. 5A, the height adjusters are presented in a closed configuration.

Reference is now made to FIG. 5B schematically presenting another side view, not to scale, of the OPEN configuration of the 3D MRD (100), rotated 90 degrees in the XZ plane with respect to FIGS. 4A and 4B, wherein the device has a substantially parallelepiped shape, with a first top main magnet (101a) and a second bottom main magnet (not shown) arranged in a face-to-face orientation along the Y axis, defining in-between a volume of interest (VOI). Each main magnet is surrounded by at least two extension-plates (103a and 103b), and at least two cage walls (not shown) confining the VOI and comprising bores (106) which comprise at least two height adjusters (105) (shown is a configuration with four such height adjusters; only two are shown). The height adjusters can be, in a non-limiting example, expandable shafts, or interlocking male and female protrusions. The height adjusters are arranged in equal groups interlocking with corresponding bores located in the extension plates (103a and 103b). In FIG. 5B, the height adjusters are presented in an open configuration.

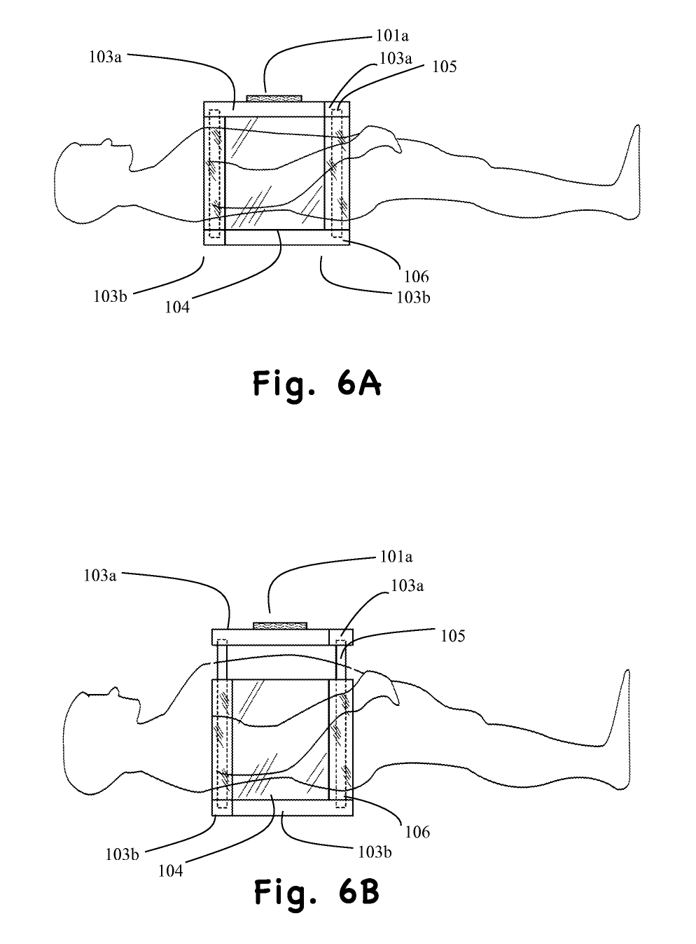

Reference is now made to FIG. 6A schematically presenting a preferred use of the MRD (100), presenting a partial side view, not to scale, with respect to an axial plane of the 3D MRD (100) wherein two main magnets (101) (only one shown) are arranged in a face-to-face orientation along the Y axis of the MRD (100). The main magnets are surrounded by extension plates (103) which are located above the cage walls (104) and the cage walls are provided with two height adjusters. The height adjusters are interlocked with corresponding bores located in the plate extensions (103) and are located in bores (106) in the outer cage walls (104) and are presented in a CLOSED configuration, defining the VOI (110) in a narrow configuration (YN) through which a patient is directed.

Reference is now made to FIG. 6B schematically presenting a preferred use of the MRD (100), presenting a partial side view, not to scale, with respect to an axial plane of the 3D MRD (100) wherein two main magnets (101) (only one shown) are arranged in a face-to-face orientation along the Y axis of the MRD (100). The main magnets are surrounded by extension plates (103) which are located above the cage walls (104) and the cage walls are provided with two height adjusters. The height adjusters are interlocked with corresponding bores located in the plate extensions (103) and are located in bores (106) in the outer cage walls (104) and are presented in an OPEN configuration, defining the VOI (110) in a wide configuration (YW) through which a wider patient is directed.

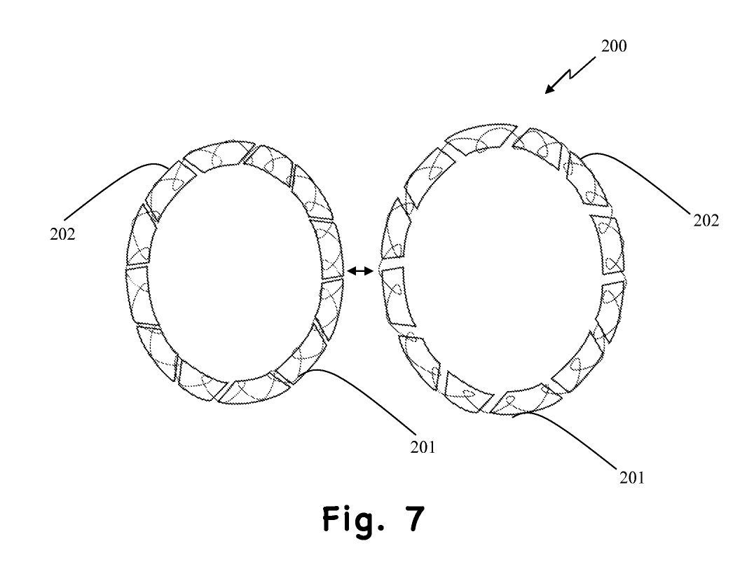

Reference is now made to FIG. 7 schematically presenting an expandable open-bore magnetic resonance device (MRD, 200) comprising at least one non-permanent superconducting electromagnet (201) coiled around the perimeter of an oval defining a magnetic volume of interest (VOI). The oval's perimeter comprises a plurality of trapezoid elements (202) housing the coil of the electromagnet. The trapezoid elements are provided with a dislocating mechanism for shifting the trapezoid elements outwards and inwards relative to the center of the oval; substantially in the plane of the oval, thus defining at least one narrow configuration (ON, left) and at least one wide configuration (OW, right) and reversibly expanding the VOI so as to enable a personalized accommodation of a user according to his dimensions. In some embodiments, adjoining trapezoid elements remain in contact along at least a portion of their sides so that the oval they form is continuous.

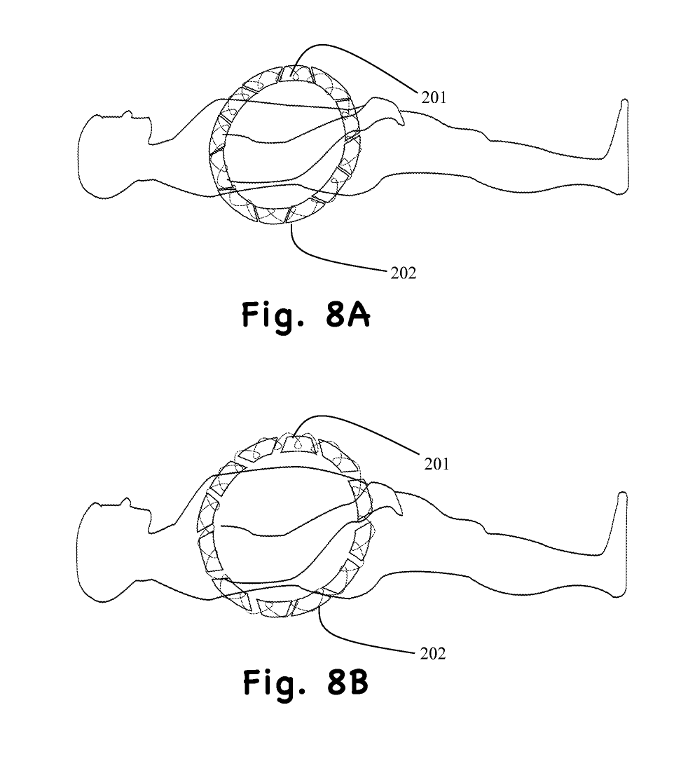

Reference is now made to FIG. 8A schematically presenting a preferable use for the expandable open-bore MRD (200). The expandable open-bore MRD (200), shown in a CLOSED configuration, comprises at least one non-permanent superconducting electromagnet (201) coiled around the perimeter of an oval defining a magnetic volume of interest (VOI). The oval's perimeter comprises a plurality of trapezoid elements (202) housing the coil of the electromagnet. The trapezoid elements are provided with dislocating means for shifting them outwards and inwards relative to the oval's center, thereby defining a VOI adapted to a user's dimensions.

Reference is now made to FIG. 8B schematically presenting a preferable use for the expandable open-bore MRD (200). The expandable open-bore MRD (200), shown in an OPEN configuration, comprises at least one non-permanent superconducting electromagnet (201) coiled around the perimeter of an oval defining a magnetic volume of interest (VOI). The oval's perimeter comprises a plurality of trapezoid elements (202) housing the coil of the electromagnet. The trapezoid elements are provided with dislocating means for shifting them outwards and inwards relative to the oval's center, thereby defining a VOI adapted to a user's dimensions.

It is hence in the scope of the invention wherein an MRD is provided with a CLOSED-configuration and an OPEN-configuration. According to an embodiment of the herein presented technology, the MRD is characterized by two or more widths (along axis Y), useful for both thin objects (such as neonates) and relatively thick objects (such as mature patients). A three-width, four-width, as well as an N-width configuration is available, according to embodiments of the invention.

It was stated that mucosal healing is a key prognostic parameter in the management of inflammatory bowel diseases (IBD), thus highlighting the role of endoscopy for monitoring of disease activity in IBD. In fact, mucosal healing has emerged as a key treatment goal in IBD that predicts sustained clinical remission and resection-free survival of patients. The structural basis of mucosal healing is an intact barrier function of the gut epithelium that prevents translocation of commensal bacteria into the mucosa and submucosa with subsequent immune cell activation. Thus, mucosal healing is considered as an initial event in the suppression of inflammation of deeper layers of the bowel wall, rather than as a sign of complete healing of gut inflammation. Anti-inflammatory or immunosuppressive drugs such as 5-aminosalicylates, corticosteroids, azathioprine, ciclosporin and anti-TNF antibodies (adalimumab, certolizumab pegol, infliximab) have been used to aid mucosal healing. However, the effectiveness of such drugs and the implications of mucosal healing for subsequent clinical management in patients with IBD are still not sufficiently well understood.

Inflammatory bowel disease (IBD) is a chronic, relapsing condition affecting the GI tract that can affect individuals of any age and results in a need for lifelong treatment, frequently including the need for surgery. (See Neurath MF1, Travis S P, Mucosal healing in inflammatory bowel diseases: a systematic review. Gut. 2012 November; 61(11):1619-35 which is incorporated herein as a reference). Hence, for example, an expandable MRD can be utilized to image a patient's abdomen or GI tract. According to this example, IBD patients are scanned to score their mucosal healing, not by a CT which has significant safety drawbacks, but by a much safer MRI device which has expandable abilities.

The invention hence discloses a method for scoring mucosal healing parameters in an IBD patient, comprising steps of providing a height (Y axis) adjustable open-bore magnetic resonance device (MRD); approaching the MRD's width to the patient's width, and scanning the patient. Moreover, the invention thus discloses means and methods for scoring IBD patients and is useful for determining, quantitatively, an IBD patient's mucosal healing score.

While the invention is susceptible to various modifications and alternative forms, specific embodiments thereof have been shown by way of example in the drawings and the above detailed description. It should be understood, however, that it is not intended to limit the invention to the particular forms disclosed, but, on the contrary, the intention is to cover all modifications, equivalents, and alternatives falling within the spirit and scope of the invention as defined by the appended claims.

What is claimed is:

1. An open-bore magnetic resonance device (MRD) having length, width and height (X, Z and Y axis, respectively), comprising:
a. a first main magnetic source and a second main magnetic source arranged in a face-to-face orientation along said Y axis, defining in-between a volume of interest (VOI);
b. at least two top extension plates extending from said first main magnetic source and at least two bottom extension plates extending from said second main magnetic source; said top and bottom extension plates are arranged in a face-to-face orientation along said Y axis; and
c. at least two outer shell walls extending perpendicular to said extension plates configured by means of size, shape and material to confine said VOI;
wherein said outer shell walls comprise a height adjuster for adjusting the distance between said first main magnetic source and said second main magnetic source; said height adjuster is provided with protruding elements for interlocking with corresponding bores in said extension plates; and said height adjusters define at least one narrow configuration (YN) and at least one wide configuration (YW), such that said VOI is reversibly expandable and a personalized accommodation of a user according to said user's dimensions is enabled.

2. The MRD according to claim 1, further comprising at least two pole-pieces, arranged in a face-to-face orientation; wherein said first main magnetic source and said second main magnetic source are permanent magnets located on said pole-pieces, arranged in a face-to-face orientation and generating a static magnetic field within said VOI.

3. The MRD according to claim 2, further comprising at least six side-magnets, at least a portion of said side-magnets are superconductors or ferromagnets; said side-magnets extend from said first and second main magnetic sources, arranged in equal groups being in a face-to-face orientation in a magnetic connection with said extension plates, such that the overall strength of the magnetic field provided in said cage is increased.

4. The MRD according to claim 3, wherein said side-magnets and said extension plates are configured as a self-fastening cage.

5. A method for obtaining an expandable cage of an MRD comprising steps of:
a. providing an open-bore magnetic resonance device (MRD) with length, width and height (X, Z and Y axis, respectively), said open-bore magnetic resonance device comprising:
i. a first main magnetic source and a second magnetic source arranged in a face-to-face orientation along said Y axis, defining in-between a volume of interest (VOI) and
ii. at least two outer shell walls for confining said VOI;
wherein, by means of two or more height adjusters comprised in said outer shell walls, reversibly adjusting the distance between said first main magnetic source and said second main magnetic source,
thereby enabling a personalized accommodation of a user according to said user's dimensions.

6. The method according to claim 5, further comprising step of providing said height adjuster with protruding elements for interlocking with corresponding bores in said outer shell walls.

7. The method according to claim 5, further comprising step of configuring said MRD as a self-fastening caged MRD, said outer shell walls comprising an outside shell.

8. The method according to claim 7, further comprising step of superimposing at least three flexi-jointed walls thereby providing a homogenous, stable and uniform magnetic field therein.

9. An expandable open-bore magnetic resonance device (MRD) comprising at least one non-permanent superconducting electromagnet coiled around a perimeter of an oval defining a magnetic volume of interest (VOI), wherein said oval's perimeter comprises a plurality of trapezoid elements housing said coil of the electromagnet, and further wherein said trapezoid elements are provided with a dislocating mechanism for shifting said trapezoid elements outwards and inwards relative to said oval's center and substantially in said oval's plane, thus defining at least one narrow configuration (ON) and at least one wide configuration (OW), thereby reversibly expanding said VOI and enabling a personalized accommodation of a user according to said user's dimensions.

10. A method for obtaining an expandable cage of an MRD comprising steps of providing an open-bore MRD comprising at least one non-permanent superconducting electromagnet, and defining a magnetic volume of interest (VOI) by coiling said electromagnet around a perimeter of an oval, wherein said method further comprises steps of providing said oval's perimeter with a plurality of trapezoid elements housing said coil of said electromagnet, and further providing said trapezoid elements with a dislocating mechanism for shifting said trapezoid elements outwards and inwards relative to said oval's center and substantially in said oval's plane, thus defining at least one narrow configuration (ON) and at least one wide configuration (OW), thereby reversibly expanding said VOI and enabling a personalized accommodation of a user according to said user's dimensions.

11. A method for scoring mucosal healing parameters in an IBD patient, comprising steps of providing an open-bore magnetic resonance device (MRD), providing at least one main magnetic source defining a volume of interest (VOI) and further providing said MRD with adjusting means for reversibly expanding said VOI, thereby enabling a personalized accommodation of said IBD patient's abdominal region according to said patient's dimensions.

12. The method according to claim 11, wherein said method defines a standard of care for said IBD patient's treatment surveillance.
